(19) United States
(12) Patent Application Publication
(10) Pub. No.: US 2023/0399646 A1
(43) Pub. Date: Dec. 14, 2023

(54) COMPOSITIONS AND METHODS FOR TREATING DISEASES OF THE LIVER

(71) Applicant: REGENTS OF THE UNIVERSITY OF MINNESOTA, Minneapolis, MN (US)

(72) Inventor: Jeffrey Holt Albrecht, Minneapolis, MN (US)

(21) Appl. No.: 18/209,039

(22) Filed: Jun. 13, 2023

Related U.S. Application Data
(60) Provisional application No. 63/352,169, filed on Jun. 14, 2022.

(51) Int. Cl. C12N 15/113; A61P 1/16

(52) U.S. Cl. CPC C12N 15/113; A61P 1/16

(57) ABSTRACT

An inhibitory nucleic molecule that targets cyclin D1 mRNA is described. Generally, the inhibitory nucleic acid molecule includes a nucleotide sequence that is complementary to at least a portion of SEQ ID NO:1. In one or more embodiments, the inhibitory nucleic acid molecule is complementary to nucleotides 330-350 of SEQ ID NO:1, nucleotides 372-392 of SEQ ID NO:1, nucleotides 525-545 of SEQ ID NO:1, nucleotides 801-825 of SEQ ID NO:1, nucleotides 1206-1230 of SEQ ID NO:1 or nucleotides 2416-2440 of SEQ ID NO:1. The inhibitory nucleic acid molecule can include RNA, DNA, or both. In another aspect, a method of treating a disease of the liver in a subject having, or at risk of having, a disease of the liver generally includes administering to the subject a composition that includes an amount of an inhibitor of cyclin D1 effective to ameliorate at least one symptom or clinical sign of the disease of the liver.

### CROSS-REFERENCE TO RELATED APPLICATION

This application claims the benefit of U.S. Provisional Patent Application No. 63/352,169, filed Jun. 14, 2022, which is incorporated herein by reference in its entirety

### GOVERNMENT FUNDING

This invention was made with government support under DK054921 awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “0110-000694US01.xml” having a size of 7 kilobytes and created on Jun. 12, 2023. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, an inhibitory nucleic acid molecule that targets cyclin D1 mRNA. Generally, the inhibitory nucleic acid molecule includes a nucleotide sequence that is complementary to at least a portion of SEQ ID NO:1.

In one or more embodiments, the inhibitory nucleic acid molecule includes a nucleotide sequence that is complementary to nucleotides 330-350 of SEQ ID NO:1, nucleotides 372-392 of SEQ ID NO:1, nucleotides 525-545 of SEQ ID NO:1, nucleotides 801-825 of SEQ ID NO:1, nucleotides 1206-1230 of SEQ ID NO:1, or nucleotides 2416-2440 of SEQ ID NO:1.

In one or more embodiments, the inhibitory nucleic acid molecule includes RNA.

In one or more embodiments, the inhibitory nucleic acid molecule includes DNA.

In another aspect, this disclosure describes a method of treating a disease of the liver in a subject having, or at risk of having, a disease of the liver. Generally, the method includes administering to the subject a composition that includes an inhibitor of cyclin D1 in an amount effective to ameliorate at least one symptom or clinical sign of the disease of the liver.

In one or more embodiments, the composition is administered before the subject manifests a symptom or clinical sign of the disease of the liver.

In one or more embodiments, the composition is administered after the subject manifests a symptom or clinical sign of the disease of the liver.

In one or more embodiments, the disease of the liver includes non-alcoholic fatty liver disease (NAFLD), non-alcoholic steatohepatitis (NASH), type 2 diabetes, metabolic syndrome, hepatic metabolic insufficiency, aging-related hepatic metabolic dysfunction, or hepatocellular carcinoma (HCC).

In one or more embodiments, the composition includes the cyclin D1 inhibitor in an amount effective to reduce hepatic steatosis, reduce hepatocyte lipotoxicity, increase lipolysis, increase lipophagy, increase autophagy, increase fatty acid oxidation, reduce hepatic inflammation in response to overnutrition, increase hepatocyte glucose uptake, increase incorporation of glucose into glycogen, reduce blood glucose levels, increase expression of hepatocyte nuclear factor 4 alpha (HNF4a) target proteins, increase expression of peroxisome proliferator-activated receptor alpha (PPARa) target proteins, increase biosynthetic and metabolic liver function, or decrease expression of senescence-associated secretory phenotype (SASP) protein expression.

In one or more embodiments, the inhibitor of cyclin D1 includes a nucleic acid molecule that is complementary to at least a portion of SEQ ID NO:1. In one or more of these embodiments, the nucleic acid molecule includes a nucleic acid sequence that is complementary to nucleotides 330-350 of SEQ ID NO:1, nucleotides 372-392 of SEQ ID NO:1, nucleotides 525-545 of SEQ ID NO:1, nucleotides 801-825 of SEQ ID NO:1, nucleotides 1206-1230 of SEQ ID NO:1, or nucleotides 2416-2440 of SEQ ID NO:1.

In one or more embodiments, the nucleic acid molecule includes RNA.

In one or more embodiments, the nucleic acid molecule includes DNA.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Acute hepatocyte-specific knockout of cyclin D1 markedly regulates metabolism after high-carbohydrate feeding. Cyclin D1^{fl/fl} mice were used to acutely knockout cyclin D1 using a hepatocyte-specific Cre vector, and control mice were treated with a GFP vector. Mice were then fasted and then refed for one day with a high-carbohydrate diet. RNA-seq was performed to assess global liver gene expression, and the Ingenuity Pathway Analysis (IPA, Qiagen) was used assess pathway regulation. (A) Western blot of liver tissue. Cyclin D1 is induced in the liver with refeeding in the control (GFP) mice, but not in the H-D1-KO mice (Cre). (B) Liver glycogen content was markedly increased in the H-D1-KO mice after refeeding.

FIG. 2. Induction of SerpinE1 (PAI-1) by cyclin D1 and aging. (A) Western blot (top) with quantification (bottom) of protein expression. Feeding induces SerpinE1 (PAI-1), but this is markedly downregulated by acute KO of cyclin D1 in hepatocytes. SerpinE1 (PAI-1) is also upregulated in aging liver. (B) Immunohistochemistry (IHC) of cyclin D1 and SerpinE1 (PAI-1) in refed mouse liver. Cyclin D1 is induced in hepatocytes in Zone 2 of the hepatic lobule. SerpinE1 is induced in hepatocytes in Zone 3, and is almost completely inhibited by KO of cyclin D1 in hepatocytes.

FIG. 3. Hepatocyte cyclin D1 expression in NASH without commensurate proliferation. Representative human liver biopsy specimens from patients with highly active autoimmune hepatitis (first column), NASH (second column), and resolved NASH after extensive weight loss following bariatric surgery (third column). Slides were immunostained for cyclin D1, as well as two markers of proliferation (Ki-67 and phospho-Histone H3). In the hepatitis patient, cyclin D1 was induced in hepatocytes, as was hepatocyte proliferation as evidenced by Ki-67 and phospho-Histone H3 staining of these cells. Substantial hepatocyte cyclin D1 expression was noted in patients with NASH and NAFLD, but this was not associated with proportionate proliferation of these cells (there was scattered positive Ki-67 and phospho-Histone H3 straining in immune cells, but positive hepatocytes were extremely rare). Resolved NASH showed no hepatocyte cyclin D1 staining, which is also the case for other normal liver human liver biopsies.

FIG. 4. Hepatocyte-specific deletion of cyclin D1 improves fatty liver in mouse NAFLD models. Normal WT mice and hepatocyte-specific cyclin D1 KO mice (H-D1-KO) were fed two different high-fat diet (HFD) models [45% fat Envigo TD.06415 and 60% fat Research Diets D12492]. (A) Western blot analysis of cyclin D1 expression and Cdk1, a marker for cell proliferation. (B) Liver triglycerides, which were lower in H-D1-KO mice.

FIG. 5. Hepatocyte expression of cyclin D1 regulates blood glucose levels in mouse models. (A) Blood glucose in mice treated with HFD as in FIG. 4. H-D1-KO mice had lower liver blood glucose levels at the time of harvest. (B) Fasting and refeeding blood glucose levels in transgenic mice with chronic Zone 2 hepatocyte overexpression of cyclin D1 (LFABP-D1) and control WT littermates, showing marked fasting hyperglycemia in the LFABP-D1 mice.

FIG. 6. Induction of cyclin D1 in aged liver is associated with decreased expression of PPARa and HNF4a target genes. (A) Livers from young (16 wk) and old (162 wk) mice were harvested for western blot for cyclin D1. (B) IHC of cyclin D1 in young and old mouse liver, demonstrating markedly increased cyclin D1 expression in hepatocytes in Zone 2 of the hepatic lobule. No evidence of hepatocyte proliferation was observed in these specimens.

### DETAILED DESCRIPTION OF ILLUSTRATIVE EMBODIMENTS

This disclosure describes compositions and methods that include RNA-interference (RNAi) and/or antisense DNA technology. The compositions may be used to inhibit expression of the cyclin D1 protein in hepatocytes, the main cell type in the liver. Inhibiting cyclin D1 in hepatocytes may be provide a therapeutic platform for treating hepatic and systemic conditions including, but not limited to, fatty liver disease, hepatic insufficiency, type 2 diabetes, and non-alcoholic steatohepatitis (NASH).

Overexpression of cyclin D1 in hepatocytes unexpectedly represses numerous metabolic functions. Further, RNAi-mediated inhibition of cyclin D1 enhances beneficial metabolic pathways in cultured hepatocytes, including glucose uptake, glycogen synthesis, lipid catabolism, fatty acid oxidation, and enhanced expression of genes and proteins that promote numerous other hepatic functions. Hepatocyte-specific knockout of cyclin D1 (H-D1-KO) in mice has numerous beneficial metabolic and anti-inflammatory effects.

This disclosure therefore describes using RNAi and/or antisense DNA to inhibit hepatocyte cyclin D1 as chronic treatment for patients with disorders such as non-alcoholic fatty liver disease (NAFLD) and its more aggressive variant non-alcoholic steatohepatitis (NASH). NASH is one cause of, for example, cirrhosis, end-stage liver disease, and hepatocellular carcinoma (HCC). At present, there are no approved drug therapies to treat NAFLD/NASH.

Cyclin D1 was originally discovered as a cell cycle protein that regulates progression through G1 phase. It is also frequently overexpressed in human cancers (including liver cancer), where it is thought to promote abnormal cell proliferation.

Cyclin D1 expression also is upregulated during normal hepatocyte proliferation. Normal hepatocyte proliferation includes liver regeneration—i.e., compensatory hepatocyte proliferation—that is an adaptive response in response to liver injury and diseases. Acute overexpression of cyclin D1 in hepatocytes is sufficient to drive proliferation of hepatocytes even under conditions that normally inhibit cell cycle progression.

Transient overexpression of cyclin D1 regulates many metabolic functions in the liver. Primarily, cyclin D1 represses many of the normal hepatic metabolic functions. Thus, transient upregulation of cyclin D1 during compensatory hepatocyte proliferation transiently decreases normal liver metabolic function, thereby allowing cellular resources to be directed to the demands of cell growth.

This disclosure describes methods that exploit findings that indicate cyclin D1 is involved in governing broad aspects of liver metabolism even in the absence of compensatory hepatocyte proliferation. That is, data provided herein show that cyclin D1 regulates hepatocyte metabolism separately and apart from its canonical role in cell cycle progression. In normal liver, little hepatocyte cyclin D1 expression is detectable. However, this disclosure provides data showing that hepatocyte cyclin D1 expression is upregulated in overnutrition, NAFLD/NASH, type 2 diabetes, aging, and cirrhosis without a commensurate increase in proliferation. The data indicate that chronically increased cyclin D1 expression regulates aspects of hepatocyte metabolism in a way that promotes liver disease including, but not limited to, fatty liver, fasting hyperglycemia, hepatic metabolic insufficiency, and inflammation. Thus, cyclin D1 plays a causative role in these disorders that makes inhibiting expression of cyclin D1 a novel therapeutic approach for treating diseases of the liver.

Generally, the therapeutic approach involves RNAi-mediated and/or antisense DNA-mediated inhibition of cyclin D1 in hepatocytes. RNAi-mediated and/or antisense DNA-mediated suppression of cyclin D1 in hepatocytes derepresses liver metabolic activity that is repressed by cyclin D1 and, therefore, restores at least a portion of liver metabolism, the loss of which promotes liver disease. Thus, while described below in the context of an exemplary embodiment in which the inhibitory nucleic acid molecule is an inhibitory RNA (RNAi) molecule, the compositions and methods described herein can involve the use of an antisense DNA molecule directed against the same targets as the described and/or exemplified RNAi molecules.

Hepatocyte cyclin D1 is upregulated by acute feeding, and broadly regulates metabolism and inflammatory pathways. Mice were fasted, then resumed feeding for one day with a high-carbohydrate diet. Cyclin D1 expression was induced in hepatocytes of refed mice without any evidence of cell proliferation. Fasting mice had virtually no expression of cyclin D1. In this model, cyclin D1 expression was acutely knocked out one week prior to the experiment using cyclin D1^{fl/fl} mice and a vector that targets the Cre recombinase specifically to hepatocytes. This model system is meant to replicate the effect of hepatocyte-specific RNAi-mediated depletion of cyclin D1 in vivo and is previously described (Wu et al., Proc. Nat. Acad. Sci. USA 117(29):17177-17186, 2020).

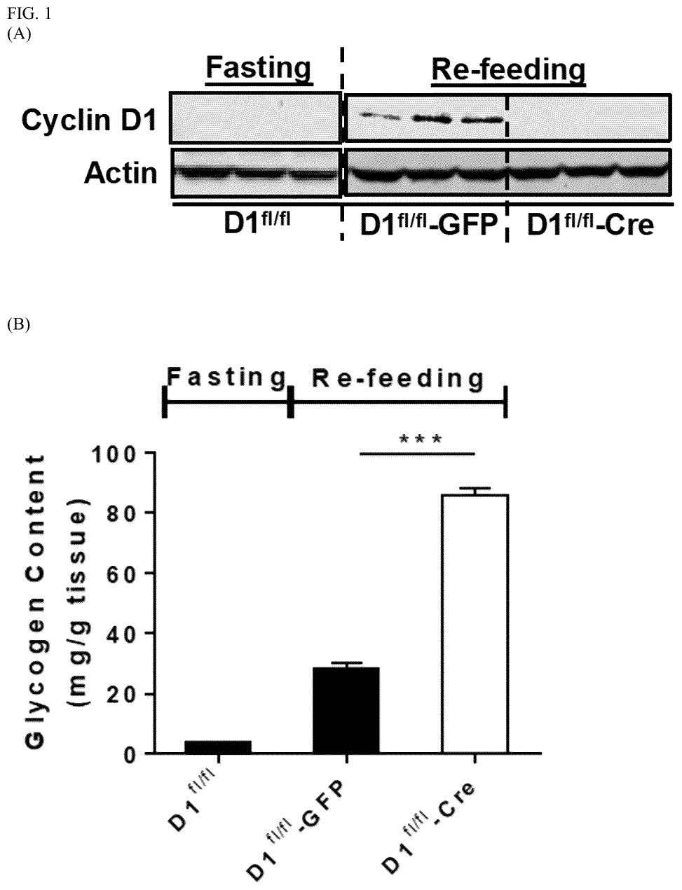

Hepatocyte-specific cyclin D1 KO markedly enhances glycogen synthesis in response to feeding. As is shown in FIG. 1A, refeeding mice a high carbohydrate diet led to induction of cyclin D1, which was not seen in the H-D1-KO mice (D1^{fl/fl}-Cre). Immunohistochemistry (IHC) studies showed that cyclin D1 was upregulated in hepatocytes in Zone 2 of the hepatic lobule. Surprisingly, H-D1-KO mice had a three-fold increase in hepatic glycogen content (FIG. 1B). This observation was confirmed in cultured hepatocytes using cyclin D1 RNAi and cyclin D1 RNAi markedly enhances hepatocyte glucose uptake (Wu et al., Proc. Nat. Acad. Sci. USA 117(29):17177-17186, 2020). Thus, inhibiting cyclin D1 expression in hepatocytes increases uptake and storage of dietary glucose in glycogen, which reflects a more favorable glucoregulatory state—i.e., hepatocytes have a greater capacity to store glucose in a “healthy” fashion. Impaired liver glucose uptake and glycogen synthesis in response to feeding is a hallmark of hepatic insulin resistance in type 2 diabetes. Thus, inhibiting cyclin D1 expression in hepatocytes (e.g., using RNAi) has substantial and potentially beneficial effects on hepatocyte glucose regulation. In addition, by diverting glucose into glycogen synthesis rather than de novo lipogenesis, this may reduce conversion of excess calories into hepatic steatosis.

Hepatocyte-specific cyclin D1 KO enhances the activity of the hepatic metabolic transcription factor PPARa after feeding. Livers from fasted and re-fed mice were analyzed under the three conditions shown in FIG. 1 using RNA sequencing (RNA-seq) to identify liver metabolic pathways regulated by cyclin D1 inhibition. RNA-seq quantifies the expression of thousands of genes, and thus provides unique insight into changes in global cell or organ functioning. Ingenuity Pathway Analysis (IPA, Qiagen, Hilden, Germany), to assess patterns of gene expression from the RNA-seq data. IPA analysis of the RNA-seq data revealed that hepatocyte-specific inhibition of cyclin D1 led to induction of PPARa target genes (Table 1), indicating that physiologically expressed cyclin D1 inhibits the activity of this metabolic transcription factor, and that inhibiting cyclin D1 expression enhances its activity, as denoted by the positive activation z-score.

TABLE 1

| Upstream regulator | Activation z-score | p-value |
|---|---|---|
| PPARa | 1.927 | 9.58 × 10⁻²² |

This study indicates that inhibiting cyclin D1 expression in hepatocytes in vivo induces the transcriptional activity of PPARa, which provide hepatic and systemic metabolic benefits. Increasing PPARa transcriptional activity, which enhances expression of genes involved in lipid breakdown and fatty acid oxidation, is one clinical goal of the hepatocyte-directed cyclin D1 RNAi therapy described herein.

Further, inhibiting hepatocyte-specific cyclin D1 expression inhibits inflammatory and acute phase response gene expression in the liver after feeding. In response to overnutrition, signals are elicited in the liver that promote the expression of genes involved inflammation. In chronic overnutrition (e.g., diet-induced obesity), this enhances activation of inflammatory cells and fibrosis in non-alcoholic steatohepatitis (NASH).

In the RNA-seq analysis of the model shown in FIG. 1, re-feeding a high-carbohydrate diet (which is a model of overnutrition) led to upregulated genes involved in hepatic inflammation and the acute phase response (APR, which is a response to injury). This was inhibited in mice with acute-hepatocyte-directed cyclin D1 knockout (Table 2 and Table 3). IPA Canonical Pathways indicating reduced hepatic inflammatory signaling with cyclin D1 knockout (Table 2). Transcripts regulated by numerous inflammatory mediators were reduced by cyclin D1 knockout (Table 3).

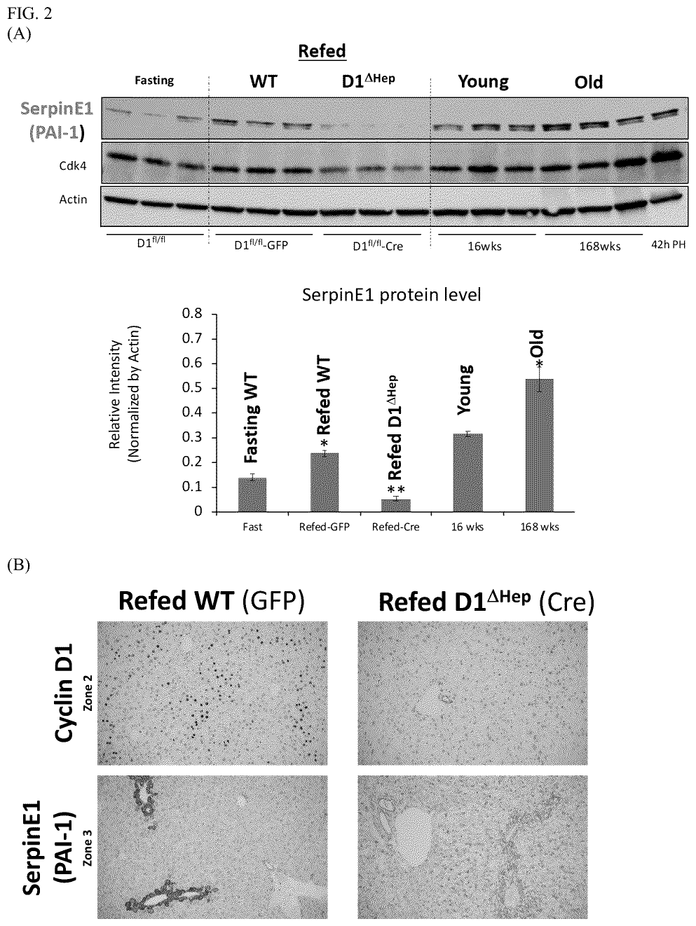

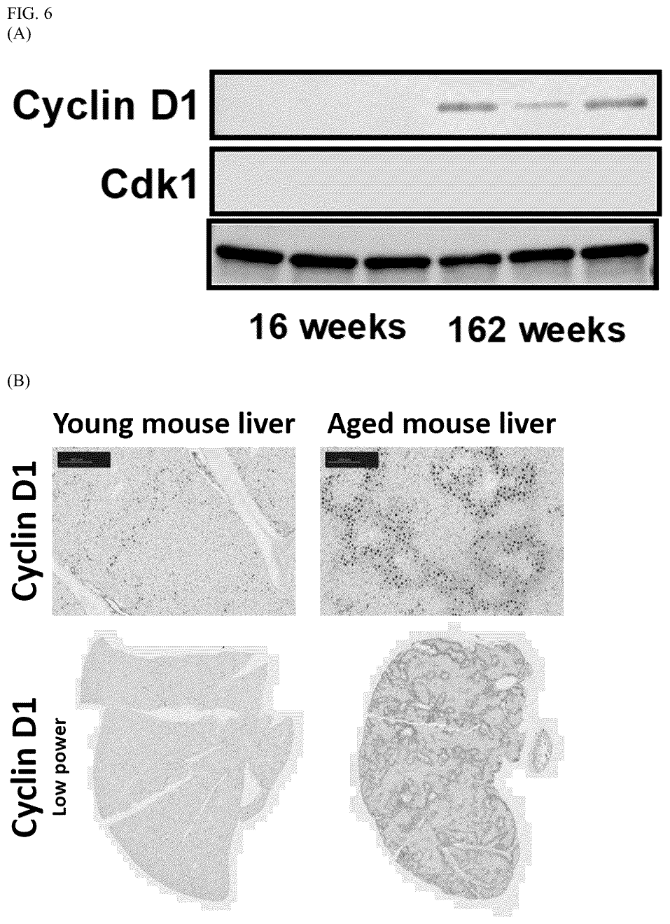

To illustrate this concept, expression of a pro-inflammatory gene, SerpinE1 (PAI-1), was examined. Expression of this protein by Western blot is shown in shown in FIG. 2A. Expression of SerpinE1 (PAI-1) is markedly inhibited by cyclin D1 KO. Interestingly, cyclin D1 is primarily expressed in Zone 2 hepatocytes, whereas SerpinE1 (PAI-1) is expressed in Zone 3 hepatocytes (FIG. 2B). These data indicate that cyclin D1 regulates proteins in distinct portions of the hepatic lobule, further supporting the concept that cyclin D1 regulates liver function. Furthermore, SerpinE1 (PAI-1) promotes NASH and aging-related phenomenon. Thus, inhibiting cyclin D1 expression markedly reduces expression of SerpinE1 (PAI-1). SerpinE1 (PAI-1) was increased in aged liver (FIG. 2A), consistent with the induction of cyclin D1 in aged liver described below (FIG. 6). The inhibition of SerpinE1 (PAI-1) by cyclin D1 inhibition is relevant because prior studies have shown that SerpinE1 (PAI-1) promotes NASH, inflammation, and aging-related changes in mouse models.

TABLE 2

| Canonical Pathway | -log(p-value) | z-score |
|---|---|---|
| Acute Phase Response Signaling | 12.8 | −3.8 |
| LPS/IL-1-Mediated Inhibition | 9.87 | −1.387 |
| of RXR Function | | |
| IL-7 Signaling Pathway | 4.33 | −0.302 |
| IL-6 Signaling | 3.95 | −1.155 |

TABLE 3

| Upstream Regulator | Activation state | Activation z-score | p-value |
|---|---|---|---|
| TNF | Inhibited | −2.095 | 1.54 × 10⁻²² |
| IL-6 | Inhibited | −5.312 | 6.28 × 10⁻²² |
| IL-1B | Inhibited | −3.335 | 6.63 × 10⁻²² |
| OSM | Inhibited | −2.484 | 3.1 × 10⁻¹⁵ |
| IL-4 | Inhibited | −3.256 | 2.38 × 10⁻¹⁰ |
| CSP2 | Inhibited | −2.502 | 8.77 × 10⁻⁸ |
| IL-1A | Inhibited | −2.323 | 8.79 × 10⁻⁸ |
| IL-17A | Inhibited | −2.931 | 4.71 × 10⁻⁷ |
| IL-22 | Inhibited | −3.196 | 8.34 × 10⁻⁶ |
| PRL | Inhibited | −2.171 | 1.17 × 10⁻⁵ |
| IL-11 | Inhibited | −2.158 | 2.51 × 10⁻⁴ |
| CNTF | Inhibited | −2.16 | 7.08 × 10⁻⁴ |
| IL-5 | Inhibited | −2.313 | 3.79 × 10⁻³ |
| IL-23A | Inhibited | −2.236 | 5.81 × 10⁻³ |
| TNFSF-12 | Inhibited | −2.377 | 0.014 |

Thus, hepatocyte expression of cyclin D1 regulates pathways related to hepatic inflammation and injury signaling in response to overnutrition. Hepatocytes themselves have immune-regulatory function and the data reported herein suggest that cyclin D1 is involved in this process, likely in a maladaptive role. Another clinical goal of the hepatocyte-directed cyclin D1 RNAi therapy described herein is reducing hepatic injury signaling and resulting fibrosis in settings with chronic cyclin D1 overexpression, such as NASH.

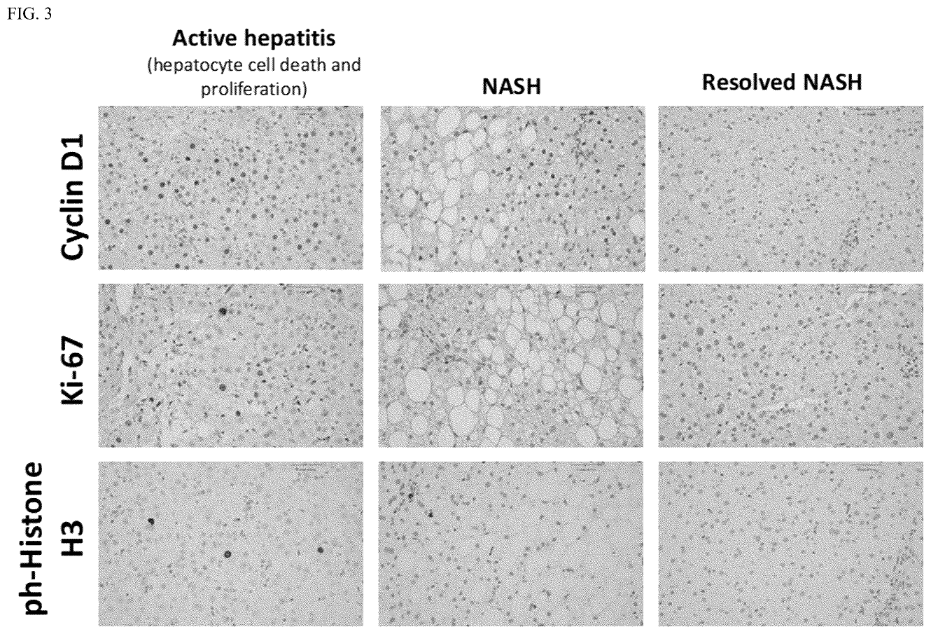

Cyclin D1 mRNA is upregulated in mouse models of NASH without evidence of proliferation. FIG. 3 shows cyclin D1 protein expression by immunohistochemistry in patients with NAFLD/NASH. In normal liver, hepatocyte cyclin D1 expression is not detectable. However, in NAFLD/NASH, numerous cyclin D1-positive hepatocytes were noted (a representative sample is shown FIG. 3). Induction of cyclin D1 in the NAFLD/NASH was not associated with histologic evidence of hepatocyte proliferation (e.g., these samples had essentially no hepatocyte Ki-67 or phospho-Histone H3). Importantly, in patients who had resolved NASH following weight loss after bariatric surgery, cyclin D1 expression was no longer present (FIG. 3). Thus, decreased cyclin D1 expression correlates with resolution of NAFLD/NASH in these clinical specimens.

These studies demonstrate that in NAFLD/NASH, hepatocyte cyclin D1 expression is upregulated in the absence of commensurate proliferation of these cells. This is consistent with data in mice that hepatocyte cyclin D1 expression in response to overnutrition and aging is uncoupled from cell cycle progression. The induction of cyclin D1 in human NAFLD/NASH provides context in support of the concept that RNAi-mediated inhibition of cyclin D1 is a viable strategy for improving fatty liver and hepatic metabolism in patients.

This disclosure therefore provides evidence that inhibiting hepatocyte cyclin D1 reduces fatty liver and improves glucose control. Mice were provided one of two different high fat diets (HFD): A 60% HFD and a 45% HFD that also had a high carbohydrate load. WT and H-D1-KO mice were compared after 10-12 weeks on the diets.

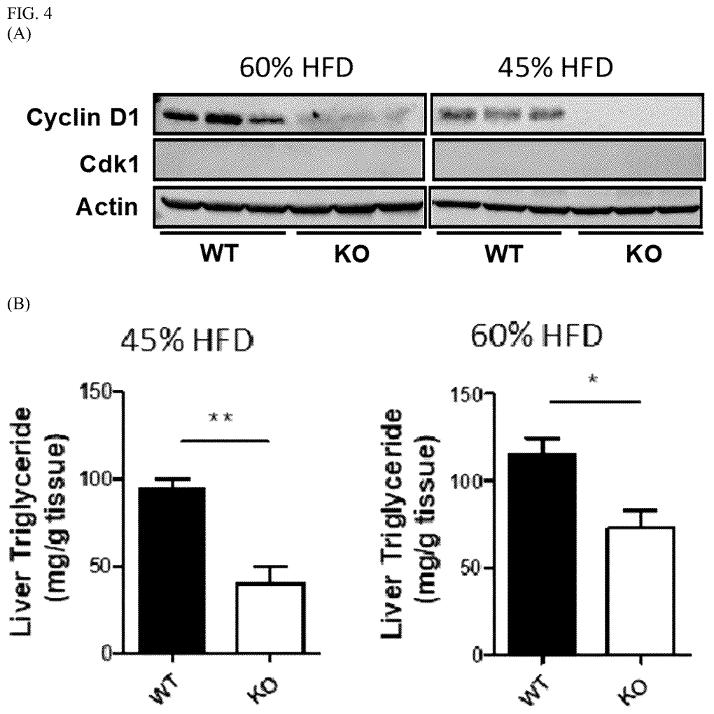

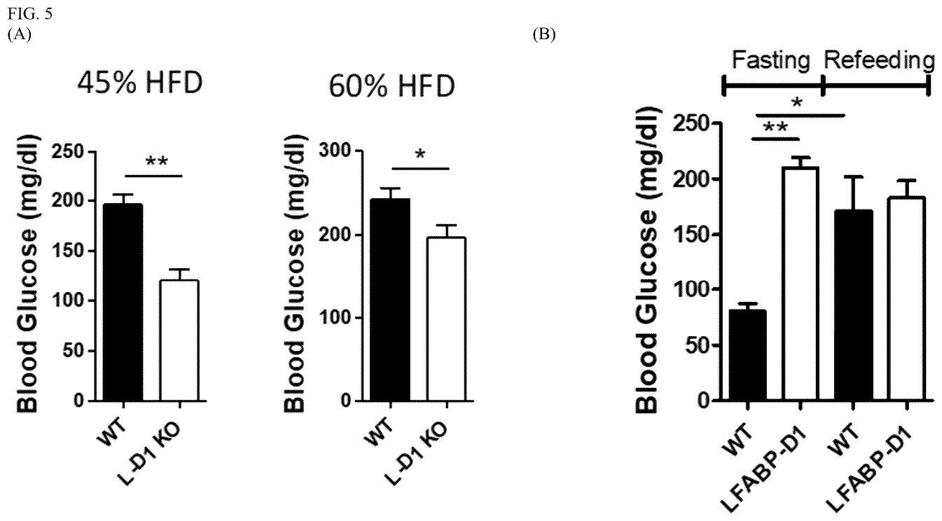

Cyclin D1 is induced by feeding a HFD, but this is not accompanied by evidence of proliferation, as evidenced by Cdk1 expression by Western Blot (FIG. 4A). Importantly, hepatocyte-specific knockout of cyclin D1 led to significantly decreased hepatic triglyceride content, providing important in vivo confirmation that reducing expression of cyclin D1 improves fatty liver (FIG. 4B). Furthermore, H-D1-KO mice had lower blood glucose levels than WT mice on the two diets (FIG. 5A).

Cyclin D1 expression was also examined is previously established transgenic mouse model of moderate chronic hepatocyte cyclin D1 expression, which has been shown to induce fatty liver as well as HCC later in life (Deane et al., 2001, Cancer Res 61:5389-5395). Immunohistochemistry analysis showed that cyclin D1 is induced in Zone 2 hepatocytes (data not shown) in this model, in a pattern very similar to that seen with overfeeding and NASH as shown in FIG. 2B and FIG. 3. In addition to promoting fatty liver, this chronic Zone 2 cyclin D1 overexpression led to marked fasting hyperglycemia (FIG. 5B; these mice are denoted as LFABP-D1). This further supports the concept that chronic expression of cyclin D1 in Zone 2 hepatocytes (similar to that documented in the settings of overnutrition, NASH, and aging) is sufficient to promote fatty liver and impaired glucose control, and that RNAi-mediated inhibition of this protein in hepatocytes is likely to be metabolically beneficial.

The data in FIG. 4 and FIG. 5 provide evidence that inhibiting physiologically-expressed hepatocyte cyclin D1 expression improves fatty liver and glucose control in vivo, which suggests the clinical efficacy of inhibiting cyclin D1 expression to treat metabolic diseases related to the liver. Inhibiting cyclin D1 expression decreases steatosis in hepatocytes by promoting lipid breakdown via lipolysis, lipophagy, and fatty acid oxidation.

Cyclin D1 inhibits the activity of HNF4a, a metabolic transcription factor in the liver, and thereby inhibits broad aspects of normal function. The transcription factor HNF4a is a well-characterized transcriptional mediator of hepatic metabolic function. Decreased activity of HNF4a is associated with type 2 diabetes and with progressive metabolic liver failure in rodents and humans with end-stage cirrhosis.

Cyclin D1 represses HNF4a transcriptional activity in hepatocytes in culture and in the liver, thereby diminishing the expression of associated genes (and their corresponding proteins) that regulate diverse metabolic activities. Hepatocyte-specific knockout of cyclin D1 enhances expression of diverse HNF4a target proteins, thereby enhancing liver metabolic function.

Thus, therapies that promote HNF4a function in hepatocytes are a promising approach to patients with liver failure. Thus, the finding that inhibiting cyclin D1 expression in hepatocytes promotes expression HNF4a transcriptional activity is a promising clinical approach to enhance liver metabolic function in advanced liver disease.

Cyclin D1 expression is increased in aging liver and corresponds with decreased PPARa and HNF4a activity. Aging is associated with decreased hepatic metabolic activity and increases the susceptibility to NAFLD/NASH and hepatocellular carcinoma (HCC), but the mechanisms are unknown. Western blot analysis and immunohistochemistry shows that cyclin D1 is diffusely induced in Zone 2 hepatocytes without evidence of commensurate proliferation (FIG. 6). IPA analysis of RNA-seq data found that the expression of PPARa and HNF4a target genes were reduced in aged liver (Table 4), which is consistent with data that cyclin D1 represses these transcription factors, as described above.

TABLE 4

| Upstream Regulator | Activation z-score | p-value |
|---|---|---|
| PPARa | −4.842 | 3.09 × 10⁻²⁶ |
| HNF4a | −3.548 | 5.95 × 10⁻¹³ |

In addition, cyclin D1 deletion in re-fed mice (using the model in FIG. 1 and FIG. 2) reduced expression of numerous hepatic genes involved in the Senescence-Associated Secretory Phenotype (SASP), such as SerpinE1 (PAI-1, FIG. 2) and CXCL14, suggesting that cyclin D1 promotes senescence-related alterations in hepatocytes.

Thus, hepatocyte cyclin D1 is induced in aging liver but not associate with increased proliferation of these cells, and this correlates with decreased activity of PPARa and HNF4a. Furthermore, cyclin D1 induces SASP proteins, suggesting that it promotes production and secretion of these proteins that adversely affect hepatic metabolism, promote inflammation, and potentially promote maladaptive effects in other organs. These data support a model in which enhanced hepatocyte cyclin D1 expression during aging reduces global hepatic metabolic function and predisposes a subject to NAFLD/NASH by repressing lipid catabolism genes downstream of PPARa and HNF4a. Thus, clinical inhibition of cyclin D1 can increase global hepatic metabolic function and decreases age-related predisposition to NAFLD/NASH.

Thus, this disclosure describes methods that generally include administering to a subject an inhibitor of cyclin D1 in an amount effective to treat a disease of the liver. Treating a disease of the liver can be prophylactic or, alternatively, can be initiated after the subject exhibits one or more symptoms or clinical signs of the condition. Treatment that is prophylactic—e.g., initiated before a subject manifests a symptom or clinical sign of the condition is referred to herein as treatment of a subject that is “at risk” of having the condition. As used herein, the term “at risk” refers to a subject that may or may not actually possess the described risk. Thus, for example, a subject “at risk” of liver disease is a subject possessing one or more risk factors associated with the liver disease such as, for example, genetic predisposition, ancestry, age, sex, geographical location, lifestyle, or medical history. Treatment may also be continued after symptoms have resolved, for example to prevent or delay their recurrence.

Accordingly, a composition that includes a cyclin D1 inhibitor can be administered before, during, or after the subject first exhibits a symptom or clinical sign of a liver disease. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, the method includes administering a composition that includes an inhibitor of cyclin D1 to a subject having, or at risk of having, a disease of the liver. In this aspect, an “effective amount” is an amount effective to reduce, limit progression, ameliorate, or resolve, to any extent, a symptom or clinical sign related to the disease of the liver.

The composition can include any suitable cyclin D1 inhibitor or combination of cyclin D1 inhibitors. In one or more embodiments, the composition can include an inhibitory RNA (RNAi) that inhibits expression of cyclin D1. Alternatively or additionally, in one or more embodiments, the composition can include an antisense DNA that inhibits expression of cyclin D1. The inhibitory nucleic acid molecule, whether an inhibitory RNA (RNAi) or an antisense DNA can be designed to include a nucleic acid sequence that is complementary to a portion of the cyclin D1 mRNA sequence (SEQ ID NO:1). Exemplary targets within the cyclin D1 mRNA sequence include, but are not limited to, nucleotides 330-350 of SEQ ID NO:1, nucleotides 372-392 of SEQ ID NO:1, nucleotides 525-545 of SEQ ID NO:1, nucleotides 801-825 of SEQ ID NO:1, nucleotides 1206-1230 of SEQ ID NO:1, or nucleotides 2416-2440 of SEQ ID NO:1. Thus, exemplary RNAi molecules include a nucleotide sequence that is complementary to nucleotides 330-350 of SEQ ID NO:1, complementary to nucleotides 372-392 of SEQ ID NO:1, complementary to nucleotides 525-545 of SEQ ID NO:1, complementary to nucleotides 801-825 of SEQ ID NO:1, complementary to nucleotides 1206-1230 of SEQ ID NO:1, or complementary to nucleotides 2416-2440 of SEQ ID NO:1.

While the nucleotide sequence of SEQ ID NO:1 is provided as an RNA sequence, a person of ordinary skill in the art can determine any complement, reverse sequence, and/or reverse complement of SEQ ID NO:1. Also, the RNA sequence of SEQ ID NO:1 can be converted to a DNA sequence by replacing each uracil with a thymine. If desired, the complementary DNA strand can be derived using conventional Watson-Crick base pairing.

The composition described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with an inhibitor of cyclin D1 without causing any undesirable biological effects or interacting in a deleterious manner with any of the other components of the pharmaceutical composition in which it is contained.

An inhibitor of cyclin D1 may therefore be formulated into a pharmaceutical composition. The pharmaceutical composition may be formulated in a variety of forms adapted to a preferred route of administration. Thus, a composition can be administered via known routes including, for example, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release. In one or more embodiments, the composition can be delivered by subcutaneous or intravenous injection.

Thus, an inhibitor of cyclin D1 may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, and the like. In one or more embodiments, the inhibitor of cyclin D1 will be formulated for delivery by subcutaneous or intravenous injection.

A formulation may be conveniently presented in unit dosage form and may be prepared by methods well known in the art of pharmacy. Methods of preparing a composition with a pharmaceutically acceptable carrier include the step of bringing the inhibitor of cyclin D1 into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of cyclin D1 inhibitor administered can vary depending on various factors including, but not limited to, the specific cyclin D1 inhibitor being administered, the weight, physical condition, and/or age of the subject, and/or the route of administration. Thus, the absolute weight of cyclin D1 inhibitor included in a given unit dosage form can vary widely, and depends upon factors such as the species, age, weight, and physical condition of the subject, and/or the method of administration. Accordingly, it is not practical to set forth generally the amount that constitutes an amount of cyclin D1 inhibitor effective for all possible applications. Those of ordinary skill in the art, however, can readily determine the appropriate amount with due consideration of such factors.

For example, certain cyclin D1 inhibitors may be administered at the same dose and frequency for which the drug has received regulatory approval. In other cases, certain cyclin D1 inhibitors may be administered at the same dose and frequency at which the drug is being evaluated in clinical or preclinical studies. In still other cases, the cyclin D1 inhibitor can be administered at a dose and/or frequency similar to the dose and/or frequency at which similarly comparable drugs have received regulatory approval. For example, compositions that include a cyclin D1 inhibitory RNA can be delivered in a manner similar to exemplary hepatocyte-directed RNAi therapies that have been approved for human use, using either GalNac-linked siRNA or lipid nanoparticles containing small interfering RNA (siRNA). Table 5 includes a non-exclusive list of exemplary hepatocyte-directed RNAi therapies.

TABLE 5

| | Gene Target | Indication | Delivery Method |
|---|---|---|---|
| Inclisiran | PCSK9 | Familial | GalNac-linked siRNA |
| | | hypercholesterolemia | (injection every |
| | | | 3-6 months) |
| Patisiran | TTR | Hereditary | Lipid nanoparticle |
| | | amyloidogenic | carrier siRNA |
| | | transthyretin | (injection every 3 weeks) |
| | | amyloidosis | |
| Givosiran | ALAS1 | Acute hepatic | GalNac-linked siRNA |
| | | porphyria | (injection once monthly) |
| Lumasiran | HAO1 | Primary hypoxaluria | GalNa-linked siRNA |
| | | type 1 | (injection every |
| | | | 1-3 months) |

Similarly, compositions that include an antisense DNA molecule can be delivered in a manner similar to exemplary antisense DNA. One can alter the dosages and/or frequency as needed to achieve a desired level of cyclin D1 inhibition. Thus, one can use standard/known dosing regimens and/or customize dosing as needed.

In one or more embodiments, the method can include administering sufficient cyclin D1 inhibitor to provide a dose of, for example, from about 100 ng/kg to about 50 mg/kg to the subject, although in one or more embodiments the methods may be performed by administering cyclin D1 inhibitor in a dose outside this range.

In some of these embodiments, the method includes administering sufficient cyclin D1 inhibitor to provide a minimum dose of at least 1 μg/kg such as, for example, at least 10 μg/kg, at least 50 μg/kg, at least 100 μg/kg, at least 200 μg/kg, at least 300 μg/kg, at least 400 μg/kg, at least 500 μg/kg, at least 600 μg/kg, at least 700 μg/kg, at least 800 μg/kg, at least 900 μg/kg, at least 1 mg/kg, at least 1.5 mg/kg, at least 2 mg/kg, at least 2.5 mg/kg, or at least 3 mg/kg.

In one or more embodiments, the method includes administering sufficient cyclin D1 inhibitor to provide a maximum dose of no more than 50 mg/kg such as, for example, no more than 25 mg/kg, no more than 20 mg/kg, no more than 15 mg/kg, no more than 10 mg/kg, no more than 5 mg/kg, no more than 3 mg/kg, no more than 1 mg/kg, or no more than 500 μg/kg.

In one or more embodiments, the method includes administering sufficient cyclin D1 inhibitor to provide a dose that falls within a range having endpoints defined by any minimum dose described above and any maximum dose described above that is greater than the selected minimum dose. For example, a dose of the cyclin D1 inhibitor may be from 300 μg/kg to 5 mg/kg, from 300 μg/kg to 3 mg/kg, from 10 μg/kg to 10 mg/kg, from 50 μg/kg to 500 μg/kg, etc.

In one or more embodiments, the dose of cyclin D1 inhibitor can be equal to any minimum dose or any maximum dose described above. Thus, for example, a dose of cyclin D1 inhibitor can be 1 μg/kg, 100 μg/kg, 300 μg/kg, 500 μg/kg, 1 mg/kg, 2.5 mg/kg, 3 mg/kg, 5 mg/kg, or 10 mg/kg.

A single dose may be administered all at once, continuously for a prescribed period of time, or in multiple discrete administrations. When multiple administrations are used, the amount of each administration may be the same or different. For example, a dose of 3 mg/kg per day may be administered as a single administration of 3 mg/kg, continuously over 24 hours, as two or more equal administrations (e.g., two 1.5 mg/kg administrations), or as two or more unequal administrations (e.g., a first administration of 2 mg/kg followed by a second administration of 1 mg/kg). When multiple administrations are used to deliver a single dose, the interval between administrations may be the same or different.

In one or more embodiments, the cyclin D1 inhibitor may be administered, for example, from a single dose to multiple doses per week, although in one or more embodiments the method can involve a course of treatment that includes administering doses of the cyclin D1 inhibitor at a frequency outside this range. When a course of treatment involves administering multiple doses within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

In one or more embodiments, the cyclin D1 inhibitor may be administered from about once per year to once per day. For embodiments in which the inhibitor of cyclin D1 is an inhibitory nucleic acid molecule, a typical dosing regimen can include providing a dose at a minimum frequency of at least once per year such as, for example, at least twice per year, at least three times per year, at least four times per year, at least five times per year, at least six times per year, at least seven times per year, at least eight times per year, at least nine times per year, at least ten times per year, at least 11 times per year, at least 12 times per year, at least 15 times per year, at least 16 times per year, at least 18 times per year, at least 20 times per year, or at least 24 times per year.

In one or more embodiments in which the inhibitor of cyclin D1 is an inhibitory nucleic acid molecule, a typical dosing regimen can include providing a dose at a maximum frequency of no more than once per day, no more than once per week, no more than twice per month, no more than once a month, no more than once every two months, no more than once every three months, no more than once every four months, or no more than once every six months.

In one or more embodiments in which the inhibitor of cyclin D1 is an inhibitory nucleic acid molecule, a typical dosing regimen can include providing a dose at a frequency that falls within a range having endpoints defined by any minimum frequency described above and any maximum frequency described above that is more frequent than the selected minimum frequency. For example, a cyclin D1 inhibitory nucleic acid molecule may be administered from once per year to once every two weeks, from twice per year to twice per month, etc.

In one or more embodiments, the dose of cyclin D1 inhibitor can administered at a frequency that is equal to any minimum frequency or any maximum frequency dose described above. Thus, for example, a dose of cyclin D1 inhibitor can be administered at a frequency of once per day, once per week, twice per month, once a month, once every three months, once every six months, or once per year.

The inhibitor of cyclin D1 can be administered for any suitable duration of time. For example, the inhibitor of cyclin D1 may be administered on an “as needed” basis. In some cases (e.g., a patient in liver failure), a single dose of cyclin D1 inhibitor may be sufficient. In other cases (e.g., a patient with a chronic condition), the treatment regimen may involve administering the inhibitor of cyclin D1 for life.

A component is said to be present in an amount “no more than” a reference amount or concentration when the component is not absent but is present in an amount up to the reference amount or concentration.

The cyclin D1 inhibitor can be administered to a subject having, or at risk of having, a disease of the liver. For example, the cyclin D1 inhibitor may be administered to treat a subject having, or at risk of having, non-alcoholic fatty liver disease (NAFLD) or its more aggressive variant non-alcoholic steatohepatitis (NASH). The cyclin D1 inhibitor may reduce hepatic steatosis (and consequent hepatocyte lipotoxicity) by promoting healthy lipid catabolism through increased lipolysis, lipophagy, and fatty acid oxidation. Alternatively, or additionally, the cyclin D1 inhibitor may reduce hepatic inflammation in response to overnutrition. Hepatic inflammation occurs in NASH and other progressive liver diseases. As another example, the cyclin D1 inhibitor may be administered to treat a subject having, or at risk of having, type 2 diabetes in the setting of NASH or the metabolic syndrome by improving hepatocyte glucose uptake, improving incorporation of glucose into glycogen, and reducing blood glucose levels in the setting of overnutrition.

In other embodiments, the cyclin D1 inhibitor may be administered to treat a subject having, or at risk of having, hepatic metabolic insufficiency in the setting of advanced cirrhosis by improving HNF4a transcriptional activity.

In other embodiments, the cyclin D1 inhibitor may be administered to treat a subject having, or at risk of having, age-related decline in hepatic metabolic function, thereby reducing the subject's age-related predisposition for developing NAFLD and/or NASH through improved HNF4a function, improved PPARa function, and/or decreased SASP expression.

In still other embodiments, the cyclin D1 inhibitor may be administered to treat a subject having, or at risk of having, hepatocellular carcinoma (HCC). Cyclin D1 is a known oncogene and is frequently overexpressed in HCC. Short-term overexpression of cyclin D1 in hepatocytes in vivo triggers certain oncogenic mechanisms that induce chromosome instability such as, for example, centrosome accumulation, abnormal mitoses, and aneuploidy. Cyclin D1 represses HNF4a—a known tumor suppressor in HCC—transcriptional activity in the liver. Thus, inhibiting chronic hepatocyte cyclin D1 expression reduces the risk of HCC.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text
SEQ ID NO: 1-Human Cyclin D1 mRNA

```
      1 AGAGGGCUGU CGGCGCAGUA GCAGCGAGCA GCAGAGUCCG CACGCUCCGG CGAGGGGCAG
    61 AAGAGCGCGA GGGAGCGCGG GGCAGCAGAA GCGAGAGCCG AGCGCGGACC CAGCCAGGAC
  121 CCACAGCCCU CCCCAGCUGC CCAGGAAGAG CCCCAGCCAU GGAACACCAG CUCCUGUGCU
  181 GCGAAGUGGA AACCAUCCGC CGCGCGUACC CCGAUGCCAA CCUCCUCAAC GACCGGGUGC
  241 UGCGGGCCAU GCUGAAGGCG GAGGAGACCU GCGCGCCCUC GGUGUCCUAC UUCAAAUGUG
  301 UGCAGAAGGA GGUCCUGCCG UCCAUGCGGA AGAUCGUCGC CACCUGGAUG CUGGAGGUCU
  361 GCGAGGAACA GAAGUGCGAG GAGGAGGUCU UCCCGCUGGC CAUGAACUAC CUGGACCGCU
  421 UCCUGUCGCU GGAGCCCGUG AAAAAGAGCC GCCUGCAGCU GCUGGGGGCC ACUUGCAUGU
  481 UCGUGGCCUC UAAGAUGAAG GAGACCAUCC CCCUGACGGC CGAGAAGCUG OGCAUCVACA
  541 CCGACAACUC CAUCCGGCCC GAGGAGCUGC UGCAAAUGGA GCUGCUCCUG GUGAACAAGC
  601 UCAAGUGGAA CCUGGCCGCA AUGACCCCGC ACGAUUUCAU UGAACACUUC CUCUCCAAAA
  661 UGCCAGAGGC GGAGGAGAAC AAACAGAUCA UCCGCAAACA CGCGCAGACC UUCGUUGCCC
  721 UCUGUGCCAC AGAUGUGAAG UUCAUUUCCA AUCCGCCCUC CAUGGUGGCA GCGGGGAGCG
  781 UGGUGGCCGC AGUGCAAGGC CUGAACCUGA GGAGCCCCAA CAACUUCCUG UCCUACUACC
  841 GCCUCACACG CUUCCUCUCC AGAGUGAUCA AGUGUGACCC GGACUGCCUC CGGGCCUGCC
  901 AGGAGCAGAU CGAAGCCCUG CUGGAGUCAA GCCUGCGCCA GGCCCAGCAG AACAUGGACC
  961 CCAAGGCCGC CGAGGAGGAG GAAGAGGAGG AGGAGGAGGU GGACCUGGCU UGCACACCCA
1021 CCGACGUGCG GGACGUGGAC AUCUGAGGGC GCCAGGCAGG CGGGCGCCAC CGCCACCCGC
1081 AGCGAGGGCG GAGCCGGCCC CAGGUGCUCC CCUGACAGUC CCUCCUCUCC GGAGCAUUUU
1141 GAUACCAGAA GGGAAAGCUU CAUUCUCCUU GUUGUUGGUU GUUUUUUCCU UUGCUCUUUC
1201 CCCCUUCCAU CUCUGACUUA AGCAAAAGAA AAAGAUUACC CAAAAACUGU CUUUAAAAGA
1261 GAGAGAGAGA AAAAAAAAAU AGUAUUUGCA UAACCCUGAG CGGUGGGGGA GGAGGGUUGU
1321 GCUACAGAUG AUAGAGGAUU UUAUACCCCA AUAAUCAACU CGUUUUUAUA UUAAUGUACU
1381 UGUUUCUCUG UUGUAAGAAU AGGCAUUAAC ACAAAGGAGG CGUCUCGGGA GAGGAUUAGG
1441 UUCCAUCCUU UACGUGUUUA AAAAAAAGCA UAAAAACAUU UUAAAAACAU AGAAAAAUUC
1501 AGCAAACCAU UUUUAAAGUA GAAGAGGGUU UUAGGUAGAA AAACAUAUUC UUGUGCUUUU
1561 CCUGAUAAAG CACAGCUGUA GUGGGGUUCU AGGCAUCUCU GUACUUUGCU UGCUCAUAUG
1621 CAUGUAGUCA CUUUAUAAGU CAUUGUAUGU UAUUAUAUUC CGUAGGUAGA UGUGUAACCU
1681 CUUCACCUUA UUCAUGGCUG AAGUCACCUC UUGGUUACAG UAGCGUAGCG UGCCCGUGUG
1741 CAUGUCCUUU GCGCCUGUGA CCACCACCCC AACAAACCAU CCAGUGACAA ACCAUCCAGU
1801 GGAGGUUUGU CGGGCACCAG CCAGCGUAGC AGGGUCGGGA AAGGCCACCU GUCCCACUCC
1861 UACGAUACGC UACUAUAAAG AGAAGACGAA AUAGUGACAU AAUAUAUUCU AUUUUUAUAC
1921 UCUUCCUAUU UUUGUAGUGA CCUGUUUAUG AGAUGCUGGU UUUCUACCCA ACGGCCCUGC
1981 AGCCAGCUCA CGUCCAGGUU CAACCCACAG CUACUUGGUU UGUGUUCUUC UUCAUAUUCU
2041 AAAACCAUUC CAUUUCCAAG CACUUUCAGU CCAAUAGGUG UAGGAAAUAG CGCUGUUUUU
2101 GUUGUGUGUG CAGGGAGGGC AGUUUUCUAA UGGAAUGGUU UGGGAAUAUC CAUGUACUUG
2161 UUUGCAAGCA GGACUUUGAG GCAAGUGUGG GCCACUGUGG UGGCAGUGGA GGUGGGGUGU
2221 UUGGGAGGCU GCGUGCCAGU CAAGAAGAAA AAGGUUUGCA UUCUCACAUU GCCAGGAUGA
2281 UAAGUUCCUU UCCUUUUCUU UAAAGAAGUU GAAGUUUAGG AAUCCUUUGG UGCCAACUGG
2341 UGUUUGAAAG UAGGGACCUC AGAGGUUUAC CUAGAGAACA GGUGGUUUUU AAGGGUUAUC
2401 UUAGAUGUUU CACACCGGAA GGUUUUUAAA CACUAAAAUA UAUAAUUUAU AGUUAAGGCU
2461 AAAAAGUAUA UUUAUUGCAG AGGAUGUUCA UAAGGCCAGU AUGAUUUAUA AAUGCAAUCU
2521 CCCCUUGAUU UAAACACACA GAUACACACA CACACACACA CACACACAAA CCUUCUGCCU
2581 UUGAUGUUAC AGAUUUAAUA CAGUUUAUUU UUAAAGAUAG AUCCUUUUAU AGGUGAGAAA
2641 AAAACAAUCU GGAAGAAAAA AACCACACAA AGACAUUGAU UCAGCCUGUU UGGCGUUUCC
2701 CAGAGUCAUC UGAUUGGACA GGCAUGGGUG CAAGGAAAAU UAGGGUACUC AACCUAAGUU
2761 CGGUUCCGAU GAAUUCUUAU CCCCUGCCCC UUCCUUUAAA AAACUUAGUG ACAAAAUAGA
2821 CAAUUUGCAC AUCUUGGCUA UGUAAUUCUU GUAAUUUUUA UUUAGGAAGU GUUGAAGGGA
2881 GGUGGCAAGA GUGUGGAGGC UGACGUGUGA GGGAGGACAG GCGGGAGGAG GUGUGAGGAG
2881 GGUGGCAAGA GUGUGGAGGC UGACGUGUGA GGGAGGACAG GCGGGAGGAG GUGUGAGGAG
2941 GAGGCUCCCG AGGGGAAGGG GCGGUGCCCA CACCGGGGAC AGGCCGCAGC UCCAUUUUCU
3001 UAUUGCGCUG CUACCGUUGA CUUCCAGGCA CGGUUUGGAA AUAUUCACAU CGCUUCUGUG
3061 UAUCUCUUUC ACAUUGUUUG CUGCUAUUGG AGGAUCAGUU UUUUGUUUUA CAAUGUCAUA
3121 UACUGCCAUG UACUAGUUUU AGUUUUCUCU UAGAACAUUG UAUUACAGAU GCCUUUUUUG
3181 UAGUUUUUUU UUUUUUUAUG UGAUCAAUUU UGACUUAAUG UGAUUACUGC UCUAUUCCAA
3241 AAAGGUUGCU GUUUCACAAU ACCUCAUGCU UCACUUAGCC AUGGUGGACC CAGCGGGCAG
3301 GUUCUGCCUG CUUUGGCGGG CAGACACGCG GGCGCGAUCC CACACAGGCU GGCGGGGGCC
3361 GGCCCCGAGG CCGCGUGCGU GAGAACCGCG CCGGUGUCCC CAGAGACCAG GCUGUGUCCC
3421 UCUUCUCUUC CCUGCGCCUG UGAUGCUGGG CACUUCAUCU GAUCGGGGGC GUAGCAUCAU
3481 AGUAGUUUUU ACAGCUGUGU UAUUCUUUGC GUGUAGCUAU GGAAGUUGCA UAAUUAUUAU
3541 UAUUAUUAUU AUAACAAGUG UGUCUUACGU GCCACCACGG CGUUGUACCU GUAGGACUCU
3601 CAUUCGGGAU GAUUGGAAUA GCUUCUGGAA UUUGUUCAAG UUUUGGGUAU GUUUAAUCUG
3661 UUAUGUACUA GUGUUCUGUU UGUUAUUGUU UUGUUAAUUA CACCAUAAUG CUAAUUUAAA
3721 GAGACUCCAA AUCUCAAUGA AGCCAGCUCA CAGUGCUGUG UGCCCCGGUC ACCUAGCAAG
3781 CUGCCGAACC AAAAGAAUUU GCACCCCGCU GCGGGCCCAC GUGGUUGGGG CCCUGCCCUG
3841 GCAGGGUCAU CCUGUGCUCG GAGGCCAUCU CGGGCACAGG CCCACCCCGC CCCACCCCUC
3901 CAGAACACGG CUCACGCUUA CCUCAACCAU CCUGGCUGCG GCGUCUGUCU GAACCACGCG
3961 GGGGCCUUGA GGGACGCUUU GUCUGUCGUG AUGGGGCAAG GGCACAAGUC CUGGAUGUUG
4021 UGUGUAUCGA GAGGCCAAAG GCUGGUGGCA AGUGCACGGG GCACAGCGGA GUCUGUCCUG
4081 UGACGCGCAA GUCUGAGGGU CUGGGCGGCG GGCGGCUGGG UCUGUGCAUU UCUGGUUGCA
4141 CCGCGGCGCU UCCCAGCACC AACAUGUAAC CGGCAUGUUU CCAGCAGAAG ACAAAAAGAC
4201 AMACAUGAAA GUOUAGAAAU AAAACUGGUA AAACCCCA
```

What is claimed is:

1. An inhibitory nucleic acid molecule that targets cyclin D1 mRNA, the inhibitory nucleic acid molecule comprising a nucleotide sequence that is complementary to at least a portion of SEQ ID NO:1.

2. The inhibitory nucleic acid molecule of claim 1, wherein the inhibitory nucleic acid molecule comprises a nucleotide sequence that is complementary to:
nucleotides 330-350 of SEQ ID NO:1;
nucleotides 372-392 of SEQ ID NO:1;
nucleotides 525-545 of SEQ ID NO:1;
nucleotides 801-825 of SEQ ID NO:1;
nucleotides 1206-1230 of SEQ ID NO:1; or
nucleotides 2416-2440 of SEQ ID NO:1.

3. The inhibitory nucleic acid molecule of claim 1, wherein the inhibitory nucleic acid molecule comprises RNA.

4. The inhibitory nucleic acid molecule of claim 1, wherein the inhibitory nucleic acid molecule comprises DNA.

5. A method of treating a disease of the liver in a subject having, or at risk of having, a disease of the liver, the method comprising administering to the subject a composition comprising an amount of an inhibitor of cyclin D1 effective to ameliorate at least one symptom or clinical sign of the disease of the liver.

6. The method of claim 5, wherein the composition is administered before the subject manifests a symptom or clinical sign of the disease of the liver.

7. The method of claim 5, wherein the composition is administered after the subject manifests a symptom or clinical sign of the disease of the liver.

8. The method of claim 5, wherein the disease of the liver comprises non-alcoholic fatty liver disease (NAFLD), non-alcoholic steatohepatitis (NASH), type 2 diabetes, metabolic syndrome, hepatic metabolic insufficiency, aging-related hepatic metabolic dysfunction, or hepatocellular carcinoma (HCC).

9. The method of claim 5, wherein the composition comprises the cyclin D1 inhibitor in an amount effective to reduce hepatic steatosis, reduce hepatocyte lipotoxicity, increase lipolysis, increase lipophagy, increase autophagy, increase fatty acid oxidation, reduce hepatic inflammation in response to overnutrition, increase hepatocyte glucose uptake, increase incorporation of glucose into glycogen, reduce blood glucose levels, increase expression of hepatocyte nuclear factor 4 alpha (HNF4a) target proteins, increase expression of peroxisome proliferator-activated receptor alpha (PPARa) target proteins, increase biosynthetic and metabolic liver function, or decrease expression of senescence-associated secretory phenotype (SASP) protein expression.

10. The method of claim 5, wherein the inhibitor of cyclin D1 comprises a nucleic acid molecule that targets cyclin D1 mRNA, the nucleic acid molecule comprising a nucleotide sequence that is complementary to at least a portion of SEQ ID NO:1.

11. The method of claim 10, wherein the nucleic acid molecule comprises a nucleic acid sequence that is complementary to:
nucleotides 330-350 of SEQ ID NO:1;
nucleotides 372-392 of SEQ ID NO:1;
nucleotides 525-545 of SEQ ID NO:1;
nucleotides 801-825 of SEQ ID NO:1;
nucleotides 1206-1230 of SEQ ID NO:1; or
nucleotides 2416-2440 of SEQ ID NO:1.

12. The method of claim 10, wherein the nucleic acid molecule comprises RNA.

13. The method of claim 10, wherein the nucleic acid molecule comprises DNA.


SEQUENCE LISTING (1 sequence)

SEQ ID NO: 1 - RNA, 4238 nt, Homo sapiens
  source 1..4238: mol_type = mRNA
agagggctgtcggcgcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagggcgccaggcaggcgggcgccaccgccacccgcagcgagggcggagccggccccaggtgctcccctgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagagaaaaaaaaaatagtatttgcataaccctgagcggtgggggaggagggttgtgctacagatgatagaggattttataccccaataatcaactcgtttttatattaatgtacttgtttctctgttgtaagaataggcattaacacaaaggaggcgtctcgggagaggattaggttccatcctttacgtgtttaaaaaaaagcataaaaacattttaaaaacatagaaaaattcagcaaaccatttttaaagtagaagagggttttaggtagaaaaacatattcttgtgcttttcctgataaagcacagctgtagtggggttctaggcatctctgtactttgcttgctcatatgcatgtagtcactttataagtcattgtatgttattatattccgtaggtagatgtgtaacctcttcaccttattcatggctgaagtcacctcttggttacagtagcgtagcgtgcccgtgtgcatgtcctttgcgcctgtgaccaccaccccaacaaaccatccagtgacaaaccatccagtggaggtttgtcgggcaccagccagcgtagcagggtcgggaaaggccacctgtcccactcctacgatacgctactataaagagaagacgaaatagtgacataatatattctatttttatactcttcctatttttgtagtgacctgtttatgagatgctggttttctacccaacggccctgcagccagctcacgtccaggttcaacccacagctacttggtttgtgttcttcttcatattctaaaaccattccatttccaagcactttcagtccaataggtgtaggaaatagcgctgtttttgttgtgtgtgcagggagggcagttttctaatggaatggtttgggaatatccatgtacttgtttgcaagcaggactttgaggcaagtgtgggccactgtggtggcagtggaggtggggtgtttgggaggctgcgtgccagtcaagaagaaaaaggtttgcattctcacattgccaggatgataagttcctttccttttctttaaagaagttgaagtttaggaatcctttggtgccaactggtgtttgaaagtagggacctcagaggtttacctagagaacaggtggtttttaagggttatcttagatgtttcacaccggaaggtttttaaacactaaaatatataatttatagttaaggctaaaaagtatatttattgcagaggatgttcataaggccagtatgatttataaatgcaatctccccttgatttaaacacacagatacacacacacacacacacacacacaaaccttctgcctttgatgttacagatttaatacagtttatttttaaagatagatccttttataggtgagaaaaaaacaatctggaagaaaaaaaccacacaaagacattgattcagcctgtttggcgtttcccagagtcatctgattggacaggcatgggtgcaaggaaaattagggtactcaacctaagttcggttccgatgaattcttatcccctgccccttcctttaaaaaacttagtgacaaaatagacaatttgcacatcttggctatgtaattcttgtaatttttatttaggaagtgttgaagggaggtggcaagagtgtggaggctgacgtgtgagggaggacaggcgggaggaggtgtgaggaggaggctcccgaggggaaggggcggtgcccacaccggggacaggccgcagctccattttcttattgcgctgctaccgttgacttccaggcacggtttggaaatattcacatcgcttctgtgtatctctttcacattgtttgctgctattggaggatcagttttttgttttacaatgtcatatactgccatgtactagttttagttttctcttagaacattgtattacagatgccttttttgtagttttttttttttttatgtgatcaattttgacttaatgtgattactgctctattccaaaaaggttgctgtttcacaatacctcatgcttcacttagccatggtggacccagcgggcaggttctgcctgctttggcgggcagacacgcgggcgcgatcccacacaggctggcgggggccggccccgaggccgcgtgcgtgagaaccgcgccggtgtccccagagaccaggctgtgtccctcttctcttccctgcgcctgtgatgctgggcacttcatctgatcgggggcgtagcatcatagtagtttttacagctgtgttattctttgcgtgtagctatggaagttgcataattattattattattattataacaagtgtgtcttacgtgccaccacggcgttgtacctgtaggactctcattcgggatgattggaatagcttctggaatttgttcaagttttgggtatgtttaatctgttatgtactagtgttctgtttgttattgttttgttaattacaccataatgctaatttaaagagactccaaatctcaatgaagccagctcacagtgctgtgtgccccggtcacctagcaagctgccgaaccaaaagaatttgcaccccgctgcgggcccacgtggttggggccctgccctggcagggtcatcctgtgctcggaggccatctcgggcacaggcccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaaggctggtggcaagtgcacggggcacagcggagtctgtcctgtgacgcgcaagtctgagggtctgggcggcgggcggctgggtctgtgcatttctggttgcaccgcggcgcttcccagcaccaacatgtaaccggcatgtttccagcagaagacaaaaagacaaacatgaaagtctagaaataaaactggtaaaacccca